(19) United States
(12) Patent Application Publication
(10) Pub. No.: US 2011/0034932 A1
(43) Pub. Date: Feb. 10, 2011

(54) SURGICAL CHISEL ASSEMBLY AND METHOD OF USE

(76) Inventor: Lonnie E. Paulos, Pensacola, FL (US)

Correspondence Address: JOHN BROOKS LAW LLC, P.O. Box 156, WRENTHAM, MA, 02093, US

(21) Appl. No.: 12/937,393

(22) PCT Filed: Oct. 21, 2008
(86) PCT No.: PCT/US08/80620

§ 371 Date: Oct. 12, 2010

Related U.S. Application Data
(60) Provisional application No. 61/045,134, filed on Apr. 15, 2008.

(51) Int. Cl. A61B 17/16

(52) U.S. Cl. 606/84

(57) ABSTRACT

Embodiments of the surgical chisel assembly comprise a wedge shaped surgical chisel designed to eliminate the square shape and sharp corners from a forward edge of a chisel. One embodiment of the chisel assembly comprises a chisel body and a grip. The chisel body comprises a wedge-shaped blade portion and a handle portion. In one embodiment, the handle portion is substantially flat about two generally parallel principal surfaces and is generally shaped to be able to be comfortably held by a surgeon in their hand. The blade portion of the chisel comprises a forward edge created by a blunting of the angle of intersection of the forward edge surfaces and a curving of the forward edge along the width of the forward edge.

### CROSS-REFERENCE TO RELATED APPLICATIONS

This application claims the benefit of U.S. App. No. 61/045,134, entitled “OSTEOTOMY CHISEL ASSEMBLY AND METHOD OF USE” and filed on 15 Apr., 2008, the entire contents of which is incorporated herein by reference.

### STATEMENT REGARDING FEDERALLY SPONSORED RESEARCH OR DEVELOPMENT:

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Technical Field

The present invention relates generally to surgical instruments and methods of their use. More particularly, embodiments of the present invention relate to new and improved osteotomes and methods of their use.

2. Background Art

In orthopedic surgery, common procedures include the use of osteotomes, or osteotomy chisel assemblies. For example, in high tibial osteotomies (HTO) osteotomes are used to cut across the bone to create wedges that help correct deformities in the tibia.

For use in these procedures, present osteotomes, or osteotomy chisel assemblies, are dangerous in that they are square tipped with sharp edges. One embodiment of the current art is disclosed in U.S. Pat. No. 6,008,433 by Kevin R. Stone. When using these embodiments, using the osteotome to cut across the bone to the opposite side, it is possible for the osteotome corners to protrude before the complete osteotomy is accomplished. These protrusions risk jeopardizing the neurovascular and ligamentous structures that are contiguous to the bone. Also, the cutting edge of the osteotome can protrude too far beyond the bone, further damaging important tissues or body structures. Even osteotomes with other shapes, such as is disclosed in U.S. Pat. No. 4,632,102 by John E. Comparetto have sharp edges to cut into the bone and present the risks associated with the sharp edges.

Other surgical procedures similarly take advantage of the features of a surgical chisel assembly. However, they are also hindered by the sharpness and the shape of the forward edge of the chisel.

### BRIEF SUMMARY OF THE INVENTION

According, one embodiment of the present invention provides a new and improved osteotome and a method of performing a surgical operation in order to correct bone deformities. In other embodiments, the chisel assembly comprises a chisel body having a wedge-shaped blade portion and a handle portion, the blade portion having a curved and blunted forward edge and the handle portion having a plurality of notches whereby the chisel assembly can be frictionally engaged by a user.

In other embodiments, the blade portion of the chisel assembly comprises two principal surfaces angularly offset by an angle of about 1 to 25 degrees.

In still other embodiments, the curved forward edge of the blade portion comprises a rounded forward edge surface connecting two principal surfaces of the blade portion.

In embodiments, the curved forward edge of the chisel assembly comprises two forward edge surfaces angularly offset by an angle of about 10 to 100 degrees and a forward edge tip surface connecting the two forward edge surfaces.

In other embodiments, the curved forward edge of the chisel assembly has a width and outside corners and the outside corners are curved at a radius of less than about ¼ of the width.

In yet other embodiments, the handle portion of the chisel assembly further comprises one or more grip portions connected to the handle portion of the chisel body.

In still other embodiments, the body portion of the chisel assembly is a rigid metal and the grip is a plastic textured material.

In embodiments, the chisel body of the chisel assembly further comprises a handle guard portion whereby the chisel assembly can be subjected to a retrograde force by applying a force to the handle guard portion.

In other embodiments, the curved forward edge of the chisel assembly comprises two forward edge surfaces angularly offset by an angle of about 10 to 100 degrees and a forward edge tip surface connecting the two forward edge surfaces, the curved forward edge has a width and outside corners, the outside corners are curved at a radius of less than about ¼ of the width, the handle portion further comprises one or more grip portions connected to the handle portion of the chisel body, the body portion is a rigid metal, the grip is a plastic textured material and the chisel body further comprises a handle guard portion whereby the chisel assembly can be subjected to a retrograde force by applying a force to the handle guard portion.

Embodiments of the present invention comprise a surgical chisel assembly kit comprising a chisel body having a wedge-shaped blade portion and a handle portion, the blade portion having a rounded and blunted forward edge and the handle portion having a plurality of notches whereby the chisel assembly can be frictionally engaged by a user.

In other embodiments, the chisel body of the surgical chisel assembly kit comprises a first chisel body having a blade portion with a width of about 1 inch and the kit further comprises a second chisel body having a blade portion with a width of about 0.5 inches.

In still other embodiments, the surgical chisel assembly kit further comprises a third chisel body having a blade portion having a width of about 1.5 inches.

Embodiments of the present invention comprise a method of wedging open a bone, the method comprising the steps of providing a surgical chisel assembly having two principal surfaces, cutting across a portion of a proximal side of the bone and applying a force to the chisel assembly whereby a blunted forward edge of the chisel assembly fractures the bone and the two principal surfaces engage and wedges open the proximal side of the bone.

In other embodiments, the method of wedging open a bone further comprises a step of securing the bone in a healing position.

In still other embodiments, the method of wedging open a bone comprises the bone further having a distal side and the step of applying a force to the chisel assembly further comprises penetrating a layer of hard cortical bone on the bone proximal and distal side but not penetrating a layer of periosteum on the distal side of the bone.

In yet other embodiments, the method of wedging open a bone includes the blunted forward edge not cutting the layer of periosteum on the distal side of the bone or a layer of one or more tissues around the bone.

### BRIEF DESCRIPTION OF THE SEVERAL VIEWS OF THE DRAWINGS

The foregoing and other features of the present invention will become more apparent upon a consideration of the following description taken in connection with the accompanying drawings wherein:

FIG. 1. A top perspective view of one embodiment of the chisel assembly.

FIG. 2. A top perspective view of one embodiment of the chisel assembly partially exploded to illustrate the body and the grip.

FIG. 3. A top view of one embodiment of the body illustrating dimensions of the body.

FIG. 4A. A side view of one embodiment of the body illustrating dimension of the body.

FIG. 4B. A side view of one embodiment of the body illustrating dimensions of the forward edge of the body.

FIG. 5. A top perspective view of one embodiment of one grip.

### DETAILED DESCRIPTION OF THE INVENTION

A surgical chisel assembly and methods for use will now be described in detail with reference to the accompanying drawings. Although embodiments are described for the use of chisels in orthopedic surgery, it is understood that the methods and systems described can be use for use in similar procedures. For example, the assemblies and methods disclosed are similarly suitable for surgical procedures involving other rigid body materials such as bone and cartilage such as may be encountered with procedures such as plastic surgery or other skeletal surgery. Notwithstanding the specific example embodiments set forth below, all such variations and modifications that would be envisioned by one of ordinary skill in the art are intended to fall within the scope of this disclosure.

### The Surgical Chisel Assembly:

Embodiments of the surgical chisel assembly comprise a wedge shaped osteotomy chisel designed to eliminate the square shape and sharp corners from a forward edge of a chisel.

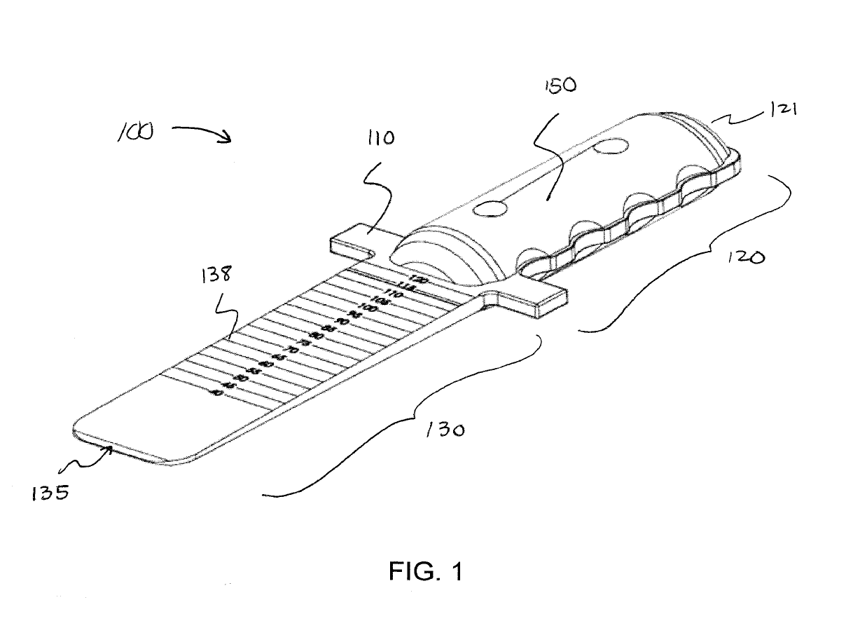

One embodiment of the chisel assembly is shown in FIG. 1 where the chisel assembly 100 comprises a chisel body 110 and a grip 150.

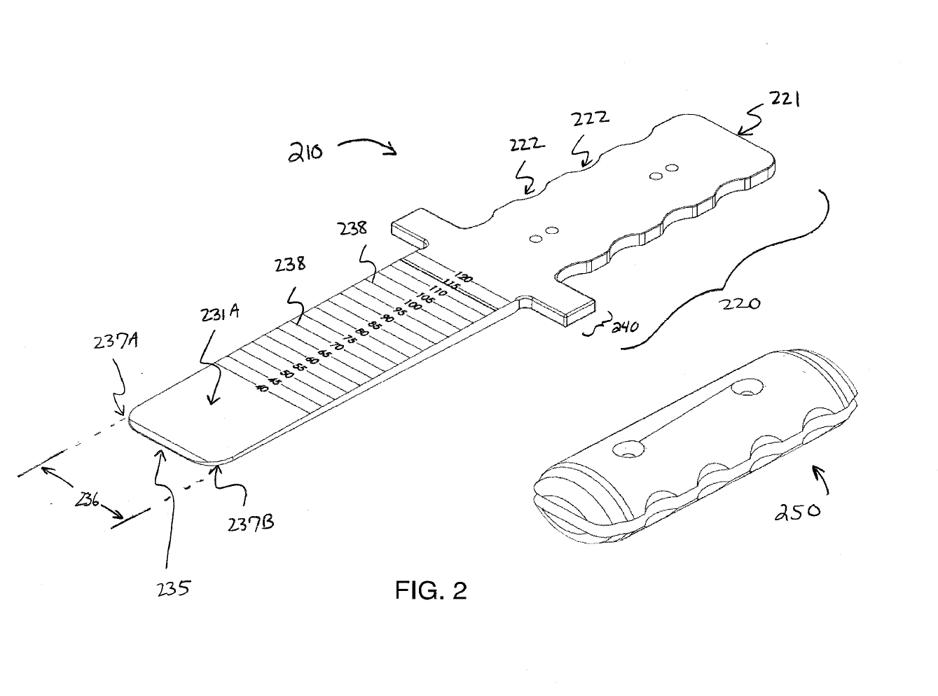

As shown in FIG. 2, the chisel body 210 comprises a wedge-shaped blade portion 230 and a handle portion 220. In this embodiment, the handle portion 220 is substantially flat about two generally parallel principal surfaces. The handle portion 220 is generally shaped to be able to be comfortably held by a surgeon in their hand. The handle portion 220 also has a generally flat proximal surface 221 that allows the end to be hit with a hand or an instrument such as a hammer. This embodiment includes notches 222 on the handle portion of the assembly to enhance the surgeon's hold of the assembly, in particular to facilitate its manual removal. In this embodiment, notches are scalloped indentations integrated into the sides of the chisel assembly but any type indentation, protrusion or texturing on the surface can be used to enhance the surgeons hold on the device. The handle portion 220 of the chisel body further includes a handle guard portion 240 representing an increase in the width dimension of the handle portion 220. The handle guard portion 240 prevents a user's hands from slipping up a blade portion 130 as well as helping the user frictionally engage the assembly and provide a protrusion to more easily apply a retrograde force by a device such as a hammer.

Referring again to FIG. 2, in this embodiment, the chisel assembly 100 has a substantially wedge-shaped blade portion 130 having two angularly offset intersecting principal surfaces 231A and 231A (not shown). The principal surfaces have a plane angle relative to each other such that the plane angle intersects at a vertex at the distal end as shown in the illustrated embodiment. The tip of the blade portion 230 defines a forward edge 235. The forward edge connects the two principle surfaces of the chisel body 210. This forward edge 235 may be defined as an extension of the principal surfaces rounded along the width of the forward edge 235. Preferably, the forward edge 235 is created by a blunting of the angle of intersection and a curving of the forward edge 235 along the width 236 of the forward edge 235.

The blunting of the forward edge 235 is defined by an increase in the intersecting surfaces of the forward edge 235 along its thickness. This creates a forward edge that is shaped more like the pointed end of a wood splitting wedge rather than the sharp edge of a knife. The curving of the forward edge 235 is defined as the rounding of the forward edge along the width 236 of the forward edge.

The unique shape of the forward edge 235 of this chisel assembly is useful in that as the chisel proceeds forward in a chisel procedure, the rounding of the forward edge 235 allow the center of the chisel's forward edge to be more likely to emerge from the bone prior to its corners 237A and 237B. Also the forward edge of the chisel assembly is blunted. Surprisingly, when the combination of features of this assembly are used to penetrate a material such as bone, properties of the bone allow the bone to fracture and crack as the blunt edge penetrates the bone, but the blunting prevents the cutting of other tissues. If the blunted forward edge engages other tissues, it is more likely to move, push or compress tissues such as muscles, ligaments or the layer of periosteum of the bone. These features prevent unwanted damage to tissues beyond the bone.

Along the blade portion 230 of the chisel assembly, calibration points 238 are included to identify the length of the tip of the forward edge 235 from the calibration points 238. For example and not for limitation, these points are etched lines on the surface 231 of the blade portion 230 at 5 millimeter increments. These points or lines allow the user to know the depth of penetration of the tip of the forward edge 235 if the distance to the forward edge is otherwise not observable.

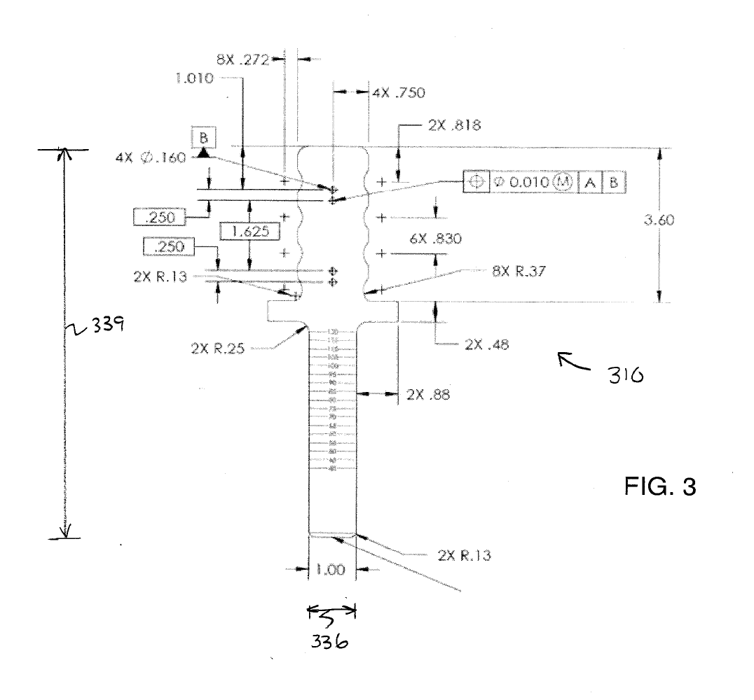

Dimensions of a body portion very similar to the embodiment illustrated in FIG. 2 are shown in FIG. 3. The dimensions in this figure represent measurements in inches. Preferably, the length 339 of the wedge-shaped body 310 ranges from about 6 to 12 inches and in the embodiment shown the length is about 8 inches. The width 336 of the blade portion is and width sufficient for the surgical use, but preferably the width ranges from about 0.25 to 2 inches and more preferably when used as a kit, from 0.5 to 1.5 inches in 0.25 inch increments. The embodiment shown in FIG. 3 has a blade portion width of 0.5 inches. The thickness of the chisel body should be sufficient to ensure the assembly is rigid when used in surgery. This thickness will vary depending of the material used to make the chisel body. When the body is made of stainless steal, for the embodiments illustrated, the handle portion of the body is about 0.2 inches thick and the blade portion tapers from this about 0.2 inches down to a thickness of 0.012 inches the forward edge. It is also contemplated that the thickness of the chisel body may be increased beyond the thickness necessary to maintain rigidity and it may be thickened to create a profile that is more useful for certain procedures. For example, in an opening wedge method of a high tibial osteotomy, an increased thickness of the blade portion may assist the surgeon in creating a larger angled opening in the bone.

As shown in FIGS. 1-3, the forward edge of the blade portion is curved about its outer edges. This curving is a rounding of the outside edges of the forward edge. As shown in FIG. 3, one embodiment of a satisfactory curve comprises a radial curve having a radius of about one eight of the width or 0.13 inches. It is understood that it is not significant that the curving be symmetrical or rounded with a consistent radius. It is also contemplated that the curving of the forward edge can be shaped parabolic or with other shaped curves that eliminate the sharp corners otherwise at the edge of the forward edge.

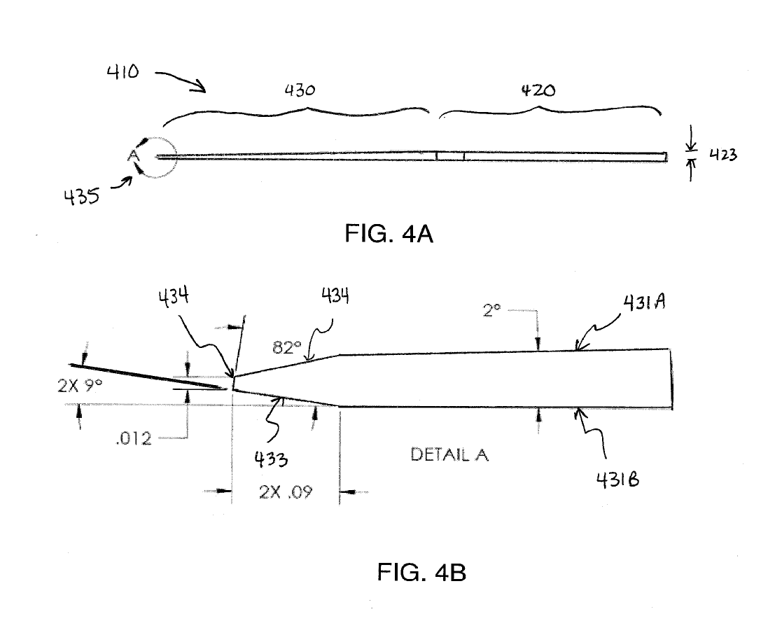

As shown in FIGS. 4A and 4B, the forward edge 435 of the blade portion 430 is also blunted about its thickness dimension 423. This blunting is an increase of the angle of the offset of the surfaces of the blade portion 430 at the forward edge 435. As shown in FIG. 4B that illustrates DETAIL A of FIG. 4A, the majority of the blade portion 430 has the surfaces 431A and 431B offset at an angle of about 2° to each other. Suitable angles for this offset can include any angles of about 1° to 25° or more preferably 1° to 12°. Nearer the forward edge 435 of the blade portion 430, this angle is altered to create the blunted edge. Preferably, this angle is altered to create an offset of the forward edges surfaces 433 and 432 of about 10° to 100°. More preferably, as in the embodiment shown, the angle changes from about 2° to about 20° at two forward edge surfaces 433 and 434 to about 91° as measured from the lower forward edge surface 433 to a tip surface 434 of the forward edge 435. Other specific angles and dimensions are also understood to provide the same blunt feature at the forward edge of the blade portion 430.

Other embodiments of the blunted edge included other angles of the forward edges or a rounding of thickness of the forward edge to create a blunted edge.

Embodiments of the chisel assembly will be provided in various dimensions. Preferably, but not necessarily, the chisel assembly will be provided with the blade portion having one-quarter inch width increments beginning with a one-half inch wide chisel and proceeding up to a one and one-half inch width.

The chisel body can be made from any surgically acceptable rigid material but preferably a stainless steel.

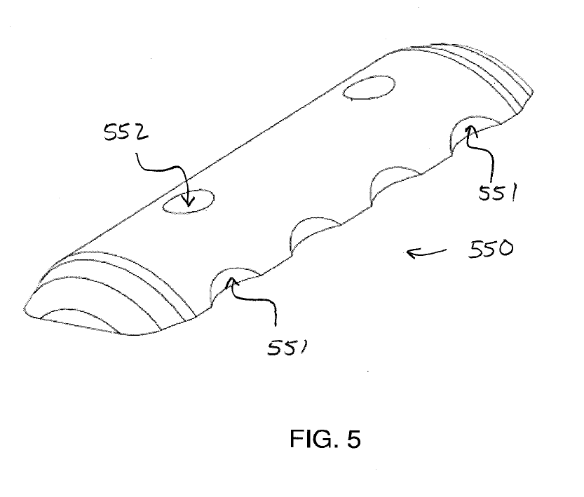

One embodiment of the chisel assembly further includes a grip connected to the handle portion. FIG. 5 illustrates a perspective view of one embodiment of one grip portion 550. When used in the chisel assembly typically two grip portions 550 (shown in FIG. 2 as 250) will be used on either side of the handle portion. The grip portions 550 are connected to the handle portion of the chisel body and have notches 551 that align with the notches of the handle portion. As illustrated, the grip portions 550 can be connected to the handle portion through screws (not shown) that fit through grip holes 552 to frictionally attach the grips portions 550 to the handle. It is also understood that the grip portions 550 can be connected to the handle with other connecting means such as adhesives, clips, hooks or other common connecting means.

Other embodiments of the grip portions include outer surfaces that help reduce the sliding of the users hand off handle while using. Examples of suitable outer surfaces for these other embodiments include, but are not limited to the outer surface having undulating shapes, finger depressions, textured and knarled surfaces or other methods to increase the frictional engagement of the user's hands.

The grip portions can be made from any rigid or material. An example of a suitable material, but not for limitation includes Radel as is sold by Solvay Advanced Polymers, LLC of Alpharetta Ga., USA.

### The Surgical Chisel Assembly in Operation:

For illustration purposes and not for limitation, the operation of the chisel assembly will be described as used in a tibial osteotomy. It is understood, that the methods described have broader applicability such as use in plastic surgery or in other surgical procedures involving the opening of bones.

As with a typical osteotomy chisel procedure use of this chisel starts with an incision being made through the patients skin just below the joint line of the knee.

Then an initial cut into the bone to be wedged open is made with a saw or a cutting chisel. In one embodiment of these methods, a power saw blade is used to make cuts through the bone. Typically, these cuts are made in locations where direct vision is possible and critical structures and other tissues are protected with retractors. In a tibial osteotomy, these cuts are typically made to the tibia anterior and lateral or medial.

Illustrating the use of the chisel assembly as illustrated in FIG. 1, the chisel assembly 100 is then used to break uncut portions of the bone and wedge apart the sides. The chisel assembly can be placed against the bone to be opened or the forward edge 135 of the chisel can be wedged into the pre-made saw cut while a force is applied to the distal end 221 of this assembly. The force is transferred to the forward edge 135 to break, expand or wedge open the tissue. For honey tissue, the chisel forces open the bone by causing the bone to fracture around the forward edge and the principal surfaces of the blade portion 130 engage the tissue and cause it to open up. In this embodiment, the inserted chisel can be used to create an opening wedge in the tibia. Cuts are made from the medial side and the chisel assembly is used to force the bone fragments apart. It is also understood that another chisel or a number of chisels with appropriate dimensions can be used to create the open wedge required for the osteotomy procedure. The chisel is inserted to the desired depth by using the calibrations 138 on the blade portion 130 to ensure the required opening is obtained. In many of these embodiments, the proximal side of the bone is fractured through a layer of hard cortical bone and a layer of cancellous bone to at least leave a portion of cortical bone in place on the distal side of the bone as a hinge. In other embodiments, such as in the case of some types of an opening type osteotomy, the hard cortical bone on both the proximal and distal side of the bone is opened but with this assembly, the distal layer of periosteum is left uncut.

With the opening wedged or otherwise opened to the appropriate dimension, bone wedges can be placed in the opening to hold them apart and the bone can then be secured in this position with the use of plates, screws or struts as typically used in osteotomy procedures.

When the tissue no longer needs to be expanded or wedged open, the chisel assembly is removed. If the chisel assembly is impacted in the tissue, the handle guard portion of the assembly can be used to loosen and remove the assembly from the tissue. If the chisel assembly requires more force to be removed, retrograde force can be applied to the handle guard to force the assembly from the tissue.

The chisel assembly can also be used in a closing wedge procedure by making 2 cuts in the tibia lateral to medial, with the cuts converging on the medial cortex. The chisel assembly is used to complete the cuts in the bone. The wedge of bone between the cuts is then removed and the bone sections are then closed together and fixed.

Although the above cuts to support opening and closing wedge procedures are described, it is understood that other types of cuts and wedge procedures can be used as well. For example, and not for limitation, a derotation cut comprising a single cut from lateral to medial can be made and the chisel assembly can break the medial side while the lower fragment is rotated with the entire lower leg inward or outward like a wheel.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Although this invention has been described in the above forms with a certain degree of particularity, it is understood that the present disclosure has been made only by way of example and numerous changes in the details of construction and combination and arrangement of parts may be resorted to without departing from the spirit and scope of the invention.

We claim:

1. A surgical chisel assembly comprising:
a chisel body having a wedge-shaped blade portion and a handle portion;
the blade portion having a curved and blunted forward edge; and
the handle portion having a plurality of notches whereby the chisel assembly can be frictionally engaged by a user.

2. The surgical chisel assembly of claim 1 wherein the blade portion comprises two principal surfaces angularly offset by an angle of about 1 to 25 degrees.

3. The surgical chisel assembly of claim 1 wherein the curved forward edge comprises a rounded forward edge surface connecting two principal surfaces of the blade portion.

4. The surgical chisel assembly of claim 1 wherein the curved forward edge comprises two forward edge surfaces angularly offset by an angle of about 10 to 100 degrees and a forward edge tip surface connecting the two forward edge surfaces.

5. The surgical chisel assembly of claim 1 wherein:
the curved forward edge has a width and outside corners; and
the outside corners are curved at a radius of less than about ¼ of the width.

6. The surgical chisel assembly of claim 1 wherein the handle portion further comprises one or more grip portions connected to the handle portion of the chisel body.

7. The surgical chisel assembly of claim 1 wherein:
the body portion is a rigid metal; and
the grip is a plastic textured material.

8. The surgical chisel assembly of claim 1 wherein the chisel body further comprises a handle guard portion whereby the chisel assembly can be subjected to a retrograde force by applying a force to the handle guard portion.

9. The surgical chisel assembly of claim 2 wherein:
the curved forward edge comprises two forward edge surfaces angularly offset by an angle of about 10 to 100 degrees and a forward edge tip surface connecting the two forward edge surfaces;
the curved forward edge has a width and outside corners;
the outside corners are curved at a radius of less than about ¼ of the width;
the handle portion further comprises one or more grip portions connected to the handle portion of the chisel body;
the body portion is a rigid metal;
the grip is a plastic textured material; and
the chisel body further comprises a handle guard portion whereby the chisel assembly can be subjected to a retrograde force by applying a force to the handle guard portion.

10. A surgical chisel assembly kit comprising:
a chisel body having a wedge-shaped blade portion and a handle portion;
the blade portion having a rounded and blunted forward edge; and
the handle portion having a plurality of notches whereby the chisel assembly can be frictionally engaged by a user.

11. The surgical chisel assembly kit according to claim 10 wherein:
the chisel body comprises a first chisel body having a blade portion with a width of about 1 inch; and
the kit further comprises a second chisel body having a blade portion with a width of about 0.5 inches.

12. The surgical chisel assembly kit according to claim 11 further comprising a third chisel body having a blade portion having a width of about 1.5 inches.

13. A method of wedging open a bone, the method comprising the steps of:
providing a surgical chisel assembly having two principal surfaces;
cutting across a portion of a proximal side of the bone; and
applying a force to the chisel assembly whereby a blunted forward edge of the chisel assembly fractures the bone and the two principal surfaces engage and wedges open the proximal side of the bone.

14. The method of claim 13 further comprising a step of securing the bone in a healing position.

15. The method of claim 13 wherein the bone further comprises a distal side and the step of applying a force to the chisel assembly further comprises penetrating a layer of hard cortical bone on the bone proximal and distal side but not penetrating a layer of periosteum on the distal side of the bone.

16. The method of claim 13 wherein the blunted forward edge does not cut the layer of periosteum on the distal side of the bone or a layer of one or more tissues around the bone.
